(19) United States
(12) Patent Application Publication
(10) Pub. No.: US 2011/0009883 A1
(43) Pub. Date: Jan. 13, 2011

(54) Vibrating hair removal device

(75) Inventor: Anand Khubani, Tawaco, NJ (US)

Correspondence Address: EPSTEIN DRANGEL LLP, 60 EAST 42ND STREET, SUITE 2410, NEW YORK, NY, 10165, US

(73) Assignee: Ideavillage Products Corp.

(21) Appl. No.: 12/459,919

(22) Filed: Jul. 9, 2009

(51) Int. Cl. A61B 17/50; A61H 1/00

(52) U.S. Cl. 606/133; 601/46

(57) ABSTRACT

The hair removal device includes an egg shape or oval base, an abrasive pad and means for securing the abrasive pad to the bottom of the base. An electrically driven vibrator of a given size and shape is removably attached to the top of the base by a resilient nesting member that defines a recess of substantially the same size and shape as the vibrator. The recess is adapted to receive and frictionally engage the vibrator. The vibrator has a substantially egg shape as does the recess such that the vibrator can be received in the nesting member in only one direction. The vibrator is driven by a battery thru a switch having a button actuator accessible from the exterior of the housing. The device includes a strap extending over the top surface of the base. The strap has ends attached to opposite edges of the base and is preferably integral with the base.

### BACKGROUND OF THE INVENTION

1. Field of the Invention

The present invention relates to hair removal devices and more particularly to a hair removal device with a removably mounted vibrator.

2. Description of Prior Art Including Information Disclosed Under 37 CFR 1.97 and 1.98

Hand-held hair removal devices of various types are known in the art. Generally, such devices include a planar base with an abrasive bottom surface and a means on the opposite (top) surface of the base for enabling the user to grasp or otherwise engage the device with his or her hand. In some instances, the engaging means takes the form of a strap spaced from the top surface of the base a sufficient distance to define a recess adapted to receive one or more fingers of the user.

The device is placed adjacent the body with the abrasive surface in contact with the skin. The device is moved in a circular motion to gently buff the skin. The motion of the device results in the removal of hair from the skin.

It would be advantageous to add a vibratory motion to the device to improve the hair removal capabilities thereof. However, to the best of applicant's knowledge, there is no simple and inexpensive hand-held hair removal device that includes an electrically driven vibrator, particularly one where the vibrator is removably mounted to the base such that it can be removed from the device to permit the device to be immersed in water for cleaning.

It is, therefore, a prime object of the present invention to provide a vibrating hair removal device.

It is another object of the present invention to provide a simple and inexpensive vibrating hair removal device.

It is another object of the present invention to provide a vibrating hair removal device which includes a removably mounted vibrator.

It is another object of the present invention to provide a vibrating hair removal device which includes a removably mounted electrically driven vibrator.

It is another object of the present invention to provide a vibrating hair removal device which includes means for removably mounting the vibrator to permit immersion of the device base in water.

It is another object of the present invention to provide a vibrating hair removal device which can be used with or without the vibrator.

### BRIEF SUMMARY OF THE INVENTION

The aforementioned objects are achieved by the present invention which relates to a hair removal device that includes a base having first and second opposing surfaces, an abrasive pad and means for securing the abrasive pad to the first base surface. Electrically driven vibrating means of a given size and shape are provided, as are means for removably attaching the vibrating means to the second base surface. The attaching means include resilient means attached to the second base surface defining a recess of substantially the same size and shape as that of the vibrating means, which is adapted to receive and frictionally engage the vibrating means.

The vibrating means has a substantially egg shaped, such that one end is narrower than the other. One end of the recess is also narrower than the other. Accordingly, the vibrating means can be received in the recess in only one direction. The recess defining means is formed of rubber-like material to enhance the frictional engagement with the vibrating means.

The base may be substantially egg shape or oval. In the egg shaped embodiment, one end of the base is narrower than the other and the narrower end of the vibrating means receiving recess extends in a direction opposite to the narrower end of the base.

The vibrating means comprises a battery, an electrically driven vibrator and a switch operably connecting the battery and the vibrator to energize the vibrator.

The device includes a strap extending over the second surface. The strap has ends attached to opposite edges of the second surface. The strap is preferably integral with the base.

### BRIEF DESCRIPTION OF THE SEVERAL VIEWS OF DRAWINGS

To these and to such other objects that may hereinafter appears, the present invention relates to vibrating hair removal device as described in detail in the following specification and recited in the annexed claims, taken together with the accompanying drawings, in which like numerals refer to like parts and in which:

FIG. 1 is an exploded perspective view of a first preferred embodiment of the present invention;

FIG. 2 is a front plan view of the device of FIG. 1;

FIG. 3 is a side plan view of the device of FIG. 1;

FIG. 4 is a top plan view of the device of FIG. 1;

FIG. 5 is a top plan view of vibrator;

FIG. 6 is a cross-sectional view of the vibrator mounting means;

FIG. 7 is an exploded lateral cross sectional view of the vibrator of FIG. 5;

FIG. 8 is a top plan view of the bottom of the vibrator casing;

FIG. 9 is a bottom plan view of the vibrator casing showing the components;

FIG. 10 is an exploded perspective view of a second preferred embodiment of the present invention;

FIG. 11 is a front plan view of the device of FIG. 10;

FIG. 12 is a side plan view of the device of FIG. 10; and

FIG. 13 is a top plan view of the device of FIG. 10.

### DETAILED DESCRIPTION OF THE INVENTION

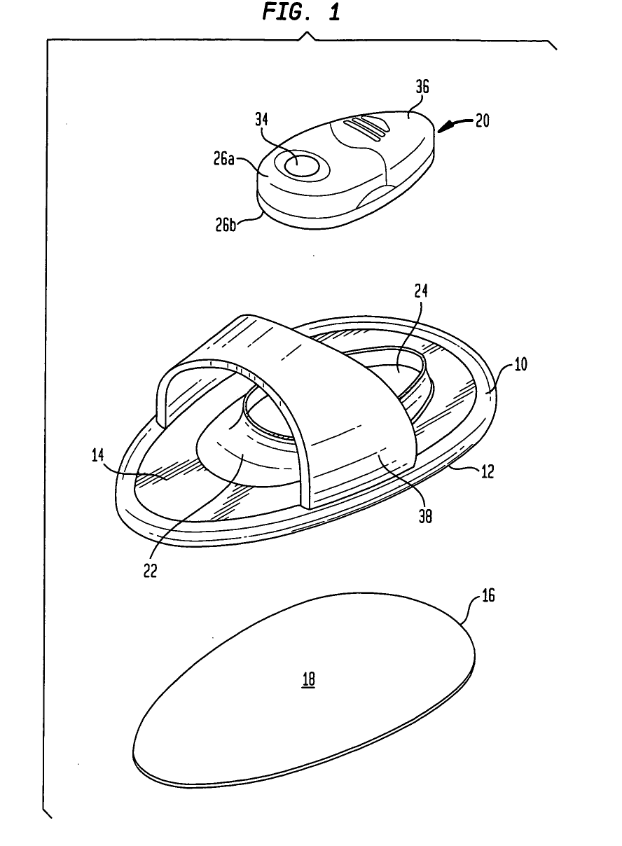

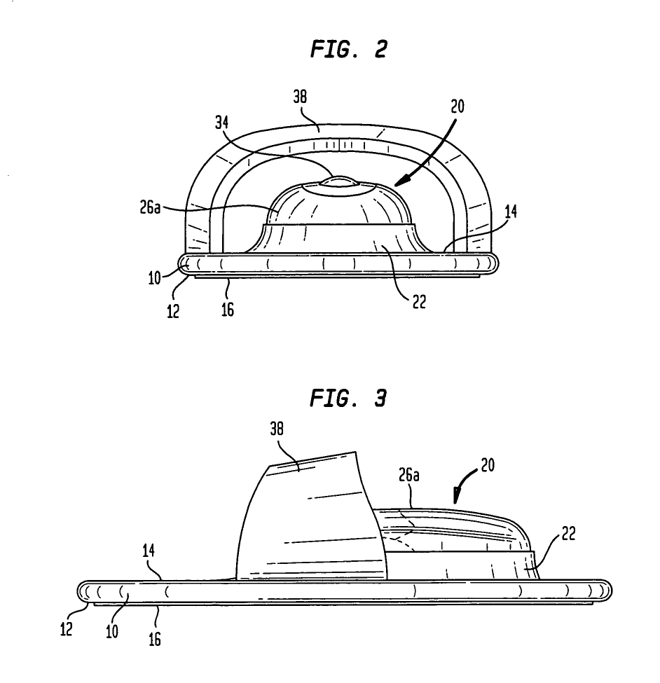

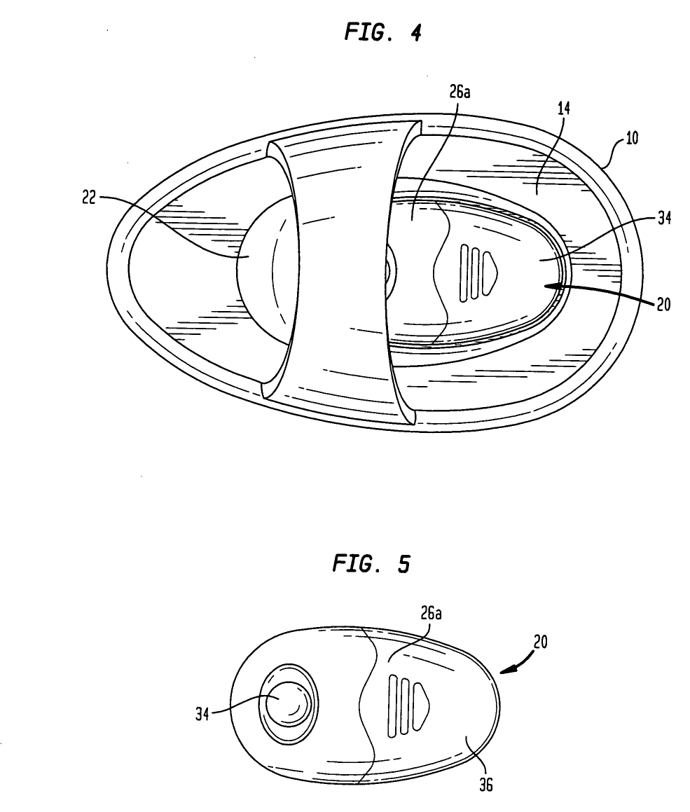

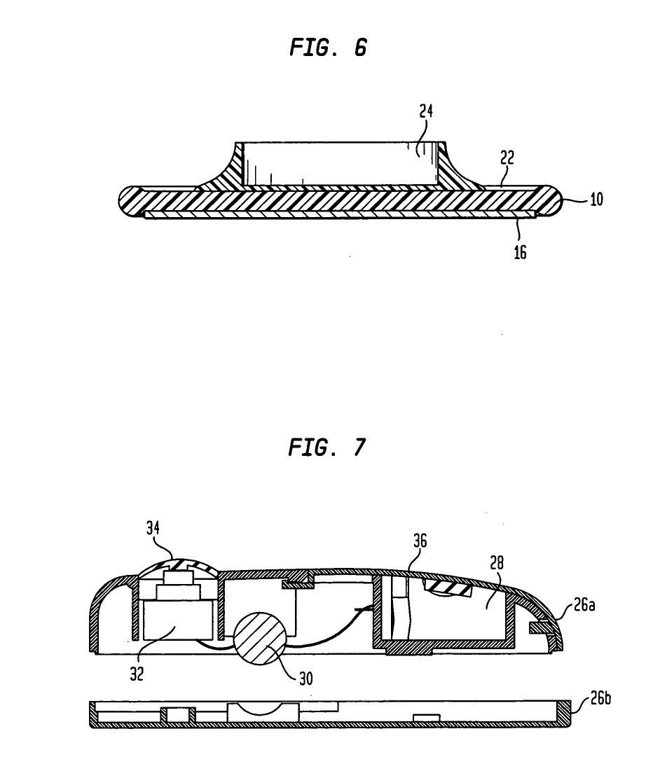

As illustrated in FIGS. 1-9, the first preferred embodiment of present invention is a hair removal device which includes a base 10 formed of resilient material such as rubber or plastic. Base 10 has a substantially planar bottom surface 12 and a substantially planar top surface 14. Base 10 is substantially egg shape, the forward end thereof being somewhat narrower that the rear end.

An abrasive pad 16 is provided having a size and shape corresponding to that of base 10. Pad 16 has a layer of adhesive 18 situated on its top surface which serves as a means for securing the abrasive pad to the bottom surface 12 of the base.

Electrically driven vibrating means, generally designated 20, are provided to be removably mounted on the top surface 14 of base 10. Permanently fixed to top surface 14 of base 10 is a resilient nesting member 22 for this purpose. Member 22 defines a recess 24 of substantially the same size and shape as that of the vibrating means 18. That recess is adapted to receive and frictionally engage the housing 26 of the vibrating means.

Housing 26 of vibrating means 20 has a substantially egg shape such that one end of the vibrating means is narrower than the other. Recess 24 also has an egg shape such that one end of recess 24 is narrower than the other. Accordingly, vibrating means 20 can be received in recess 24 in only a single direction. Member 22 is formed of rubber-like material such that it can securely frictionally engage the exterior of the housing of vibrating means 20 in a removable manner. The narrower end of recess 24 extends in a direction opposite to the narrower end of the base 10.

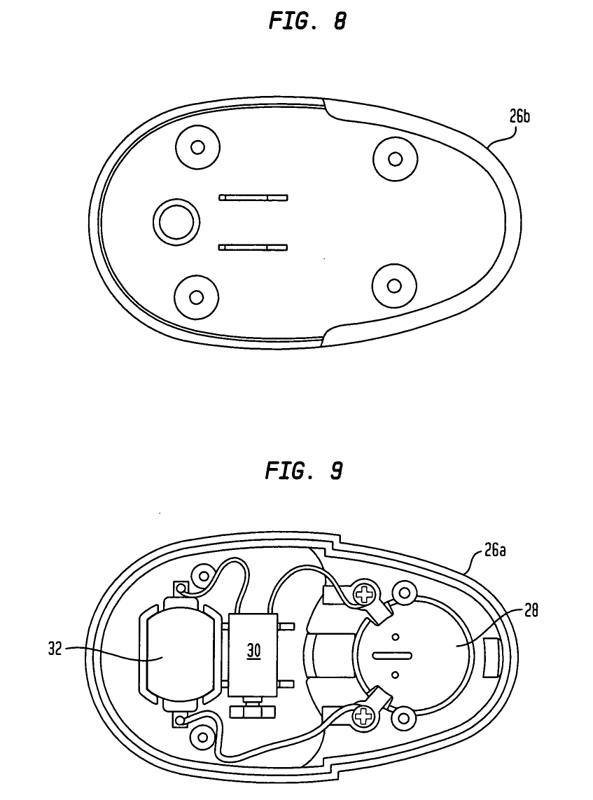

As best seen in FIGS. 7, 8 and 9, housing 26 of vibrating means 20 includes an upper or top part 26a and a lower or bottom part 26b. Housing parts 26a and 26b engage to enclose a battery receiving recess 28, an electrically driven mechanical vibrator unit 30 and a switch 32 operably connecting a battery (not shown) and vibrator 30 to energize the vibrator. Switch 32 has a push button actuator 34 accessible from the top surface of the vibrating means housing to turn the switch on and off. Upper housing part 26a has a removable battery access member 36 to permit replacement of the battery.

The device includes a strap 38 extending over and spaced from the top surface 14 of base 10. The ends of strap 38 are attached to opposite edges of the top surface 14 of the base. Strap 38 is formed of flexible material. The strap is preferably integral with the base.

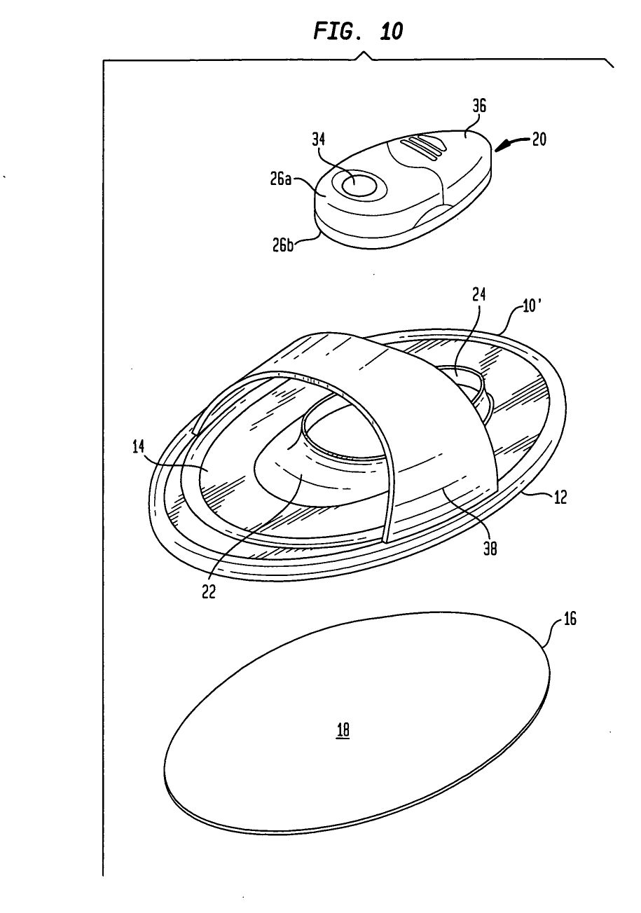

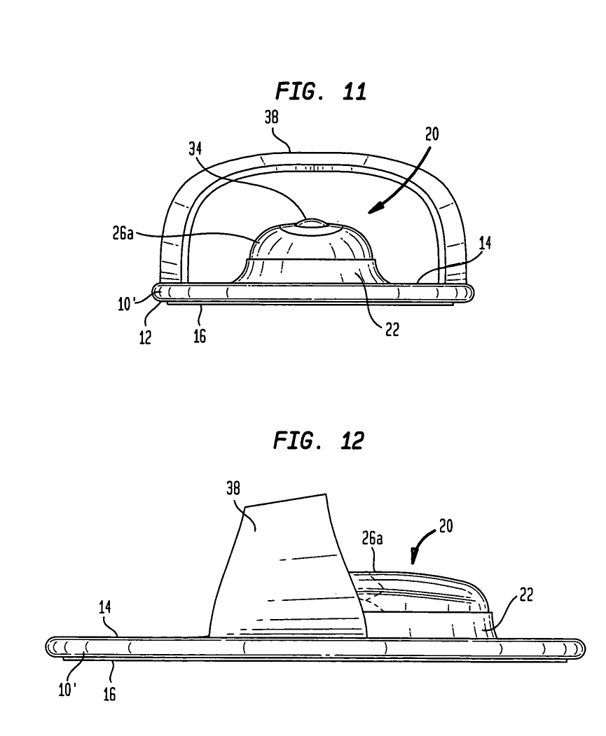

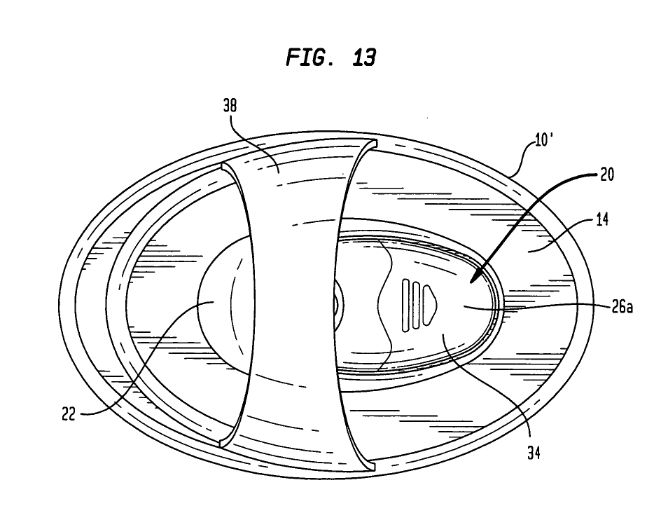

FIGS. 10 through 13 illustrate a second preferred embodiment of the vibrating hair removal device of the present invention. The second preferred embodiment of the present invention is structurally and functionally identical in every material way to the first preferred embodiment except that in the second preferred embodiment, the shape of base 10′ is oval, such that both ends of the base are of substantially equal width.

While only a limited number of preferred embodiments of the present invention have been disclosed for purposes of illustration, it is obvious that many modifications and variations could be made thereto. It is intended to cover all of those modifications and variations which fall within the scope of the present invention, as defined by the following claims.

I claim:

1. A hair removal device comprising a base having first and second opposing surfaces, an abrasive pad, means for securing said abrasive pad to said first surface, electrically driven vibrating means of a given size and shape, and means for removably attaching said vibrating means to said second surface, said attaching means comprising nesting means attached to said second surface defining a recess of substantially the same size and shape as said vibrating means adapted to receive and frictionally engage said vibrating means.

2. The device of claim 1 wherein said vibrating means has a substantially egg shape.

3. The device of claim 1 wherein said vibrating means comprises a battery, an electrically driven vibrator and a switch operably connecting said battery and said vibrator.

4. The device of claim 1 wherein said nesting means is formed of rubber-like material.

5. The device of claim 1 further comprising a strap extending over said base.

6. The device of claim 5 wherein said strap has ends attached to opposite edges of said base.

7. The device of claim 5 wherein said strap is integral with said base.

8. The device of claim 1 wherein said base is substantially egg shape.

9. The device of claim 1 wherein said base is substantially oval.

10. The device of claim 1 wherein one end of said base is narrower than the other.

11. The device of claim 10 wherein one end of said recess is narrower than the other.

12. The device of claim 11 wherein the narrower end of said recess extends in a direction opposite to the narrower end of said base.

13. The device of claim 1 wherein said vibrating means can be received in said recess in only one direction.
